(19) United States
(12) Patent Application Publication
(10) Pub. No.: US 2009/0042930 A1
(43) Pub. Date: Feb. 12, 2009

(54) PHARMACEUTICAL COMPOSITIONS CONTAINING CLOPIDOGREL BISULFATE

(75) Inventors: Jeong KU, Yongin-city (KR); Dong-Kwon LIM, Seongnam-city (KR); Eun-Young YANG, Suwon-city (KR); Tae-Kun AN, Yongin-city (KR); Eun-Kyung JEON, Yongin-city (KR); Kwang-Do CHOI, Anyang-city (KR); Yong-Sik YOUN, Yongin-city (KR); Tae-Hyoung KIM, Changwon-city (KR); Hea-Ran SUH, Icheon-city (KR); Chang-Ju KIM, Seoul (KR)

Correspondence Address: CANTOR COLBURN, LLP, 20 Church Street, 22nd Floor, Hartford, CT, 06103, US

(73) Assignee: CJ CHEILJEDANG CORP., Seoul (KR)

(21) Appl. No.: 11/995,646

(22) PCT Filed: Jul. 14, 2006
(86) PCT No.: PCT/KR2006/002779

§ 371 Date: Jan. 14, 2008

(30) Foreign Application Priority Data
Jul. 14, 2005 (KR) 10-2005-0063768

(51) Int. Cl. A61K 31/4365; A61P 7/00

(52) U.S. Cl. 514/301

(57) ABSTRACT

Provided is a pharmaceutical composition including clopidogrel bisulfate and a pregelatinized starch. The pharmaceutical composition in which the stability of clopidogrel bisulfate is significantly enhanced can be obtained by mixing clopidogrel bisulfate with the pregelatinized starch.

### TECHNICAL FIELD

The present invention relates to pharmaceutical compositions containing clopidogrel bisulfate, and in particular, to a pharmaceutical composition containing clopidogrel bisulfate to improve the stability of clopidogrel.

### BACKGROUND ART

Clopidogrel is a dextro-rotatory enantiomer of methyl alpha-5-(4,5,6,7-tetrahydro (3,2-c) thieno pyridyl) (2-chlorophenyl)-acetate, which is disclosed in U.S. Pat. No. 4,847,265. According to U.S. Pat. No. 4,847,265, clopidogrel is useful as a medicine for prophylaxis and the treatment of thromboembolism, such as thrombosis, or myocardial infarction, by acting as a platelet aggregation inhibitor.

EP 281459 discloses clopidogrel bisulfate prepared to improve stability and solubility of clopidogrel. However, EP 281459 does not disclose the polymorphism of polymorphic crystalline forms of clopidogrel bisulfate.

However, it was discovered in U.S. Pat. No. 6,429,210 that clopidogrel bisulfate can exist in different polymorphic crystalline forms which differ from each other in terms of stability, physical properties, spectral characteristics and the process by which the different polymorphic crystalline forms are prepared. In U.S. Pat. No. 6,429,210, the clopidogrel bisulfate disclosed in EP 281459 is named Crystalline Form I and a novel polymorph sulfate disclosed in U.S. Pat. No. 6,429,210 is named Crystalline Form II. In addition, a method of preparing the novel polymorph sulfate is disclosed in U.S. Pat. No. 6,429,210. According to U.S. Pat. No. 6,429,210, the powder of Crystalline Form II is more compact and much less electrostatic than Crystalline Form I and may hence have better formulation processibility. In particular, clopidogrel bisulfate in its polymorphic Crystalline Form II is thermodynamically more stable than Crystalline Form I. Since such thermodynamic stability results in a delay of decomposition of medicines over time, Plavix®, which is a commercially available clopidogrel bisulfate, contains Crystalline Form II according to U.S. Pat. No. 6,429,210 as an active ingredient.

However, in the method of preparing Crystalline Form II disclosed in U.S. Pat. No. 6,429,210, the mother liquors from which Crystalline Form I are obtained yield Crystalline Form II after a 3 to 6 months period. With respect to the method of preparing Crystalline Form II disclosed in U.S. Pat. No. 6,429,210, Crystalline Form II of clopidogrel bisulfate requires substantially more time and efforts than Crystalline Form I. Despite such problems of requiring more time and effects, Crystalline Form II is still commercially used due to high stability in medicine over time resulting from its thermodynamic stability. In the present application, “stability” refers to a tendency that a relative amount of impurities and/or decomposed products that can be generated during formulation and/or storage is minimized.

However, if the use of Crystalline Form I of clopidogrel bisulfate guarantees stability equal to or higher than when Crystalline Form II is used, there is no reason to use Crystalline Form II of clopidogrel bisulfate for which a manufacturing period is 3 or more months longer than Crystalline Form I. Accordingly, in this case, the use of Crystalline Form I of the clopidogrel bisulfate may be advantageous in terms of time and effort.

### DESCRIPTION OF THE DRAWING

FIG. 1 is a graph illustrating the results of the dissolution test on tablets prepared according to Examples 1 and 2 and Comparative Examples 1 through 4, according to an embodiment of the present invention.

### DETAILED DESCRIPTION OF THE INVENTION

### Technical Problem

The inventors of the present application studied clopidogrel-containing pharmaceutical compositions in which clopidogrel bisulfate has a high stability and completed the invention disclosed in the present application.

Accordingly, the present invention provides clopidogrel-containing pharmaceutical compositions capable of improving the stability of clopidogrel bisulfate.

### Technical Solution

According to an aspect of the present invention, there is provided a pharmaceutical composition comprising clopidogrel bisulfate and pregelatinized starch.

In the pharmaceutical composition, the amount of clopidogrel bisulfate can be in the range of 30-40 parts by weight, and the amount of the pregelatinized starch can be in the range of 10-70 parts. In addition, the clopidogrel bisulfate-containing pharmaceutical composition may further comprise an additive which is conventionally used in the technical field of pharmaceutics.

In the clopidogrel bisulfate-containing pharmaceutical composition, the clopidogrel bisulfate can be Crystalline Form I or Crystalline Form II. When the clopidogrel bisulfate is Crystalline Form I, stability equal to or higher than when commercially available Crystalline Form II is used can be obtained. When the clopidogrel bisulfate is Crystalline Form II, a higher stability can be obtained than when commercially available Crystalline Form II is used.

The gelatinized starch of the pharmaceutical composition can be any pregelatinized starch. For example, the pregelatinized starch can be selected from the group consisting of pregelatinized corn starch, prepotato starch, pregelatinized wheat starch, and a mixture thereof.

The pharmaceutical composition can be formulated in a solid-phase preparation having various shapes. For example, the pharmaceutical composition can be formulated in a various solid-phase pharmaceutical preparation in the form of, particularly, granules, tablets, or capsules.

Hereinafter, the present invention will be described in detail.

Inventors of the present application conducted research to improve the stability of clopidogrel bisulfate and discovered that when clopidogrel bisulfate is formulated by mixing clopidogrel bisulfate with pregelatinized starch, the stability of clopidogrel bisulfate substantially increases. Accordingly, the present invention provides a pharmaceutical composition comprising clopidogrel bisulfate and pregelatinized starch capable of improving the stability of clopidogrel bisulfate.

In the pharmaceutical composition, the amount of clopidogrel bisulfate may be in the range of 30-40 parts by weight and the amount of the pregelatinized starch may be in the range of 10-70 parts by weight. When the amount of clopidogrel bisulfate and pregelatinized starch are within these ranges, the clopidogrel bisulfate can be effectively stable. However, the amounts of clopidogrel bisulfate and the pregelatinized starch are not limited thereto. When the amount of pregelatinized starch is greater than 10 parts by weight, the stability of the clopidogrel bisulfate can substantially increase. However, the preferred amount of the pregelatinized starch is in the range of 15-20 parts by weight.

The pregelatinized starch can improve the stability of the clopidogrel bisulfate, which is contained in the pharmaceutical composition and can control the speed of release of the medicine.

The pregelatinized starch has a molecular formula of (C₆H₁₀O₅)ₙ and the molecular weight of the pregelatinized starch is in the range of 300-1000. The pregelatinized starch has higher flowability and compressibility than a starch so that it has been used as a binder in a dry tableting process. In some cases, the pregelatinized starch can be used together with a diluent and a lubricant. The lubricant can be magnesium stearate but according to the amount thereof, the hardness or ejection can deteriorate. Accordingly, in general, a stearic acid or sodium stearyl fumarate is used as the lubricant. The pregelatinized starch can also be used during a process of manufacturing wet granules, and has a moisture content of 18-23%. The pregelatinized starch has hygroscopicity and is stored in a tightly closed container in a dark, cold chamber. Starch 1500 having a low moisture content contains moisture of 7% or less and generally used to formulate into a capsule.

It has been reported that when the pregelatinized starch, such as Starch 1500, is alone used with aspirins that can be affected by moisture, or when the pregelatinized starch and microcrystalline cellulose (MCC), such as Avicel, is used with aspirins, the stability of aspirins can increase. This is because pregelatinized starch inherently is inclined to retain 10-15% of moisture therein so that the starch can remove all the moisture of a preparation and protect the primary components from moisture itself and/or a substance affected by moisture (The Effect of STARCH 1500 On The Stability of Aspirin Pellets Stored Under Accelerated Conditions Charles R et al, Colorcon, West Point, Pa., USA AAPS, October, 2001).

As such, the inventors of the present application added the pregelatinized starch to clopidogrel bisulfate and observed an increase in the stability of the clopidogrel bisulfate. As described above, although the pregelatinized starch is known to improve the stability of a medicine that is sensitive to moisture, clopidogrel bisulfate is not a substance that is sensitive to moisture and there were no attempts to improve the stability of Crystalline Form I of clopidogrel bisulfate by adding the pregelatinized starch to clopidogrel bisulfate despite a need to improve the stability of Crystalline Form I of clopidogrel bisulfate. However, the inventors of the present application discovered that when the pregelatinized starch is added to clopidogrel bisulfate during a process of formulating clopidogrel bisulfate, Crystalline Form I of clopidogrel bisulfate has a stability equal to or greater than Crystalline Form II of clopidogrel bisulfate, and the stability of Crystalline Form II substantially increases.

The pregelatinized starch used in the embodiment of the present invention can be any pregelatinized starch. For example, the pregelatinized starch can be selected from the group consisting of pregelatinized corn starch, such as Starch 1500, pregelatinized potato starch, pregelatinized wheat starch, and a mixture thereof. Particularly, the pregelatinized starch may be Starch 1500 which is a pregelatinized corn starch having a low moisture content.

The composition including clopidogrel bisulfate and pregelatinized starch according to an embodiment of the present invention can be formulated into a solid preparation. According to the kind of the dosage form which is formulated, various additives that are commonly used in the field of pharmaceutics may be further added to the composition comprising clopidogrel bisulfate and pregelatinized starch.

An additive, which is to be further added to the composition, may be different according to the dosage form which is formulated. For example, the composition may further include a conventional additive selected from the group consisting of a diluent, a lubricant, a disintegrant, a binder, etc.

The diluent can be selected from the group consisting of a microcrystalline cellulose (MCC), such as Avicel; dextrose; starch; sucrose; lactose; sorbitol; mannitol; calcium phosphate, such as bicalcium, tricalcium; and a mixture thereof. However, the diluent is not limited thereto. When the diluent is the MCC, the amount of MCC may be in the range of 10-90 wt %, preferably 20-40 wt %, based on the total weight of the unit dosage formulation.

The lubricant can be selected from the group consisting of light anhydrous silicic acid; metallic stearate, such as magnesium stearate; talc; staric acid; sodium stearyl fumarate; hydrogenanated vegetable oil; wax having a high melting point; and a mixture thereof, but the lubricant is not limited thereto. The amount of lubricant may be in the range of 0.2-2 wt %, but preferably about 0.75 wt %, based on the total weight of the unit dosage formulation.

The disintegrant can be starch glycolic sodium, such as Primojel; starch; alginic acid or a sodium salt thereof; talc; corn starch; or a mixture thereof.

The binder can be polyvinylpyrrolidone, magnesium aluminum silicate, starch paste, gelatin, tragacanth, methylcellulose, sodium carboxy methylcellulose, hydroxypropylcellulose, copovidone, or a mixture thereof. A solvent used to prepare the tablets can be water, ethanol, or lower alcohols, such as isopropanol.

The composition according to an embodiment of the present invention may further include, in addition to the additives described above, other additives commonly used in the field of pharmaceutics depending on the dosage form which is formulated. For example, other additives may include an azotropic mixture, an absorbing agent, a coloring agent, a flavoring agent, or a sweetening agent.

The composition according to an embodiment of the present invention is preferably formulated into tablets. A method of formulating the composition into tablets can be a wet granulation method, a dry granulation method, or a direct compression method. The amount of clopidogrel bisulfate, which is an active component, is 75 mg per unit which is in the range of 30-40 wt % based on the total weight of the conventional tablets, and the degree of mixing does not need to be considered when the composition is mixed.

Accordingly, the pharmaceutical composition according to an embodiment of the present invention can be prepared by simply mixing an active component, Starch 1500, and Avicel PH102, and then additionally mixing the mixture with a lubricant, such as assodium stearyl fumarate. The prepared pharmaceutical composition is then formulated into a tablet.

The tablet formed using the pharmaceutical composition may be covered by a film coating layer. The film coating layer can be formed of any polymer that can form a film coating layer. The amount of polymer used may be as low as possible in order to control the size of the tablet and to effectively prepare the tablet. The amount of polymer may be in the range of about 1-10 wt %, preferably about 3-5 wt %, based on the total amount of the formulation.

The coating can be performed according to a conventional coating method. In particular, the coating may be performed using aqueous coating by a pan coating method used to coat tablets. For example, in aqueous coating by a general pan coating method, commercially available opadry AMB (Aqueous Moisture Barrier), which is a coating agent including 45.52% of PVA (polyvinyl alcohol) and can be obtained from Colorcon Co., is suspended in water in order to prepare a coating solution, and then a pan coater, such as a Hi-coater, is filled with the coating solution. Then, the coating is performed at a influx temperature of 50 to 80° C. and an discharged air temperature of about 30-45° C. The coated product is dried using a conventional method, such as a drying method using dry air for 30 minutes, in order to form a film coating layer.

The pharmaceutical composition according to an embodiment of the present invention may further include, in addition to the additives used for the formulation, a stabilizer known in the art to hinder the decomposition of an active component, as required. The stabilizer can be an antioxidant, such as ascorbyl palmitate, ascorbyl stearate; an aqueous chelating agent, such as sodium ethylenediaminetetracetic acid (EDTA), sodium ascorbate; or the like.

### Advantageous Effects

As described above, according to the present invention, a clopidogrel bisulfate-pharmaceutical composition in which the stability of clopidogrel bisulfate is significantly enhanced can be obtained by mixing clopidogrel bisulfate with a pregelatinized starch.

### Best Mode

The present invention will be described in further detail with reference to the following examples. These examples are for illustrative purposes only and are not intended to limit the scope of the present invention.

### EXAMPLE

### Examples 1 and 2, and Comparative Examples 1 through 4

(1) Preparation of Tablets

Tablets were prepared using active components and additives in composition ratios according to Examples 1 and 2 and Comparative Examples 1 through 4 illustrated in Table 1, respectively. In each of Examples 1 and 2 and Comparative Examples 1 through 3, all the components, but except of sodium stearyl fumarate, were mixed in a composition ratio illustrated in Table 1 using a mixer, and then sodium stearyl fumarate was added thereto and completely mixed. The mixture was compressed using a rotary press (Korsch PH106) in order to be formulated into 100,000 white tablets. The weight of a unit tablet was 248 mg.

TABLE 1

| | Weight (mg) | | | | | |
|---|---|---|---|---|---|---|
| | Comparative | Example | Comparative | Comparative | Example | Comparative |
| Composition | Example 1 | 1 | Example 2 | Example 3 | 2 | Example 4 |
| Crystalline Form | 97.875 | 97.875 | 97.875 | 97.875 | 97.875 | 97.875 |
| 1 of clopidogrel | | | | | | |
| bisulfate | | | | | | |
| Avicel | 147.625 | 91.925 | 91.925 | 91.925 | 84.425 | 31.0 |
| Starch 1500 | | 55.7 | | | 55.7 | |
| Mannitol | | | 55.7 | | | 68.925 |
| PEG | | | | | | 34.0 |
| Hydrogenated | | | | | | 3.3 |
| castor oil | | | | | | |
| L-HPC | | | | | | 12.9 |
| Kollidon VA 64 | | | | 55.7 | | |
| Crosscarmelose | | | | | 7.5 | |
| sodium | | | | | | |
| Sodium steryl | 2.5 | 2.5 | 2.5 | 2.5 | 2.5 | |
| fumarate | | | | | | |
| Weight | 248 | 248 | 248 | 248 | 248 | 248 |

In Comparative Example 4, an active component, mannitol, avicel, PEG, and L-HPC were mixed in a composition ratio illustrated in Table 1, and then granulated using hydrogenated caster oil. Then, the resultant granules were collected through an 18 mesh sieve, and then dried using an air flow dryer at a temperature in a range of 40 to 45° C. The dried product was uniformly arranged through a 20 mesh sieve and then compressed using a rotary press (Korsch PH 106) in order to produce 100,000 white tablets. The weight of a unit tablet was 248 mg. The pellet prepared according to Comparative Example 4 is the same as a commercially available Plavix formulated using Crystalline Form II of clopidogrel bisulfate, except that Crystalline Form I was used instead of Crystalline Form II of clopidogrel bisulfate.

(2) Preparation and Coating of Coating Suspension

Each of the prepared tablets were loaded to a coating pan (Hi-coater) and the discharge air temperature was maintained at a temperature in the range of about 30 to 40° C. 12 g of opadry AMB (obtained from Colorcon Co.) coating agent was suspended in 48 g of water in order to prepare a coating solution. The coating solution was sprayed onto the dry tablets using a spray operating by air pressure, and then dried with air for about 10 minutes. The amount of the obtained coated layer was 4.83% of each pellet. The average weight of each pellet was in the range of 256-264 mg.

### Experimental Example 1: Dissolution Test

Comparative Dissolution Tests on tablets prepared according to Examples 1 and 2 and Comparative Examples 1 through 4 and a Plavix, which is commercially available from Sanofi-synthelabo Co., were carried out.

Dissolution Tests were carried out according to Dissolution Test Article 2 of General Test of Korea Pharmacopoeia. The conditions of a buffer solution are 37° C., 50 rpm, and a pH of 2.0. The samples were collected 15, 30, and 60 minutes after the dissolution tests were initiated. The analysis of the samples was carried out by chromatography under the conditions as shown in Table 2.

TABLE 2

| | |
|---|---|
| Column | Stainless steel column filled with ULTRON |
| | ES-OVM in a stationary phase (length: 0.15 |
| | m, inner diameter: 4.6 mm) |
| Detector | Absorptiometic detector (220 nm) |
| Flow Rate | 0.7 mL/minutes |
| Dosage | 10.0 μl |
| Mobile Phase | Solution S1:Acetonitril = 80:20 |
| | (Solution S1 was prepared by dissolving 1.36 |
| | g of KH₂PO₄ with 500.0 mL of water and then |
| | making the total amount of the obtained |
| | solution to 1000.0 mL) |

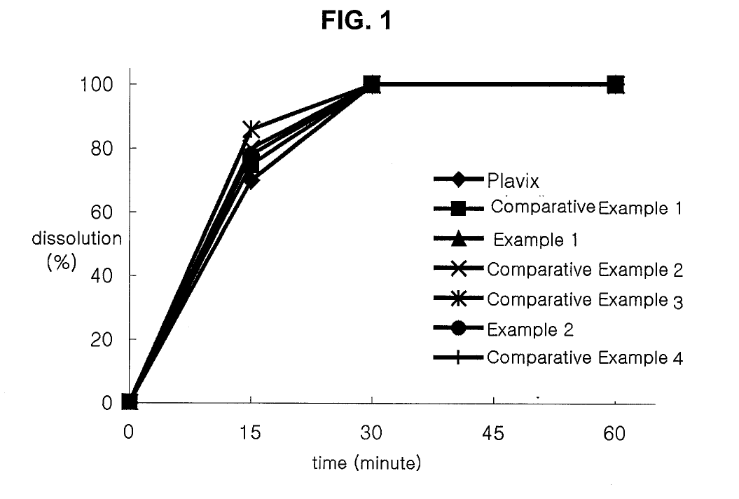

The results obtained from the dissolution tests are shown in Table 3 and FIG. 1.

TABLE 3

| | Dissolution Ratio (%) | | | | | | |
|---|---|---|---|---|---|---|---|
| | | Comparative | | Comparative | Comparative | | Comparative |
| Time (min) | Plavix | Example 1 | Example 1 | Example 2 | Example 3 | Example 2 | Example 4 |
| 0 | 0 | 0 | 0 | 0 | 0 | 0 | 0 |
| 15 | 70 | 75 | 80 | 94 | 86 | 78 | 73 |
| 30 | 100 | 100 | 100 | 100 | 100 | 100 | 100 |
| 60 | 100 | 100 | 100 | 100 | 100 | 100 | 100 |

As shown in Table 3 and FIG. 1, it was discovered that 30 minutes after the dissolution initiation, about 80% or more of the active ingredients contained in the tablets prepared according to Examples 1 and 2 and Comparative Examples 1 through 4 were released.

### Experimental Test 2: Stability Test

The tablets prepared according to Examples 1 and 2 and Comparative Examples 1 through 4 and Plavix (obtained from Sanofi-Synthelabo Co.) were stored at 60° C.(relative humidity of 80%) for 4 weeks and the contents (%) of the active ingredients contained in the respective tablets were measured.

In the respective cases, 20 tablets were crushed into powder and then mixed. Then, 260.0 mg of the powder mixture was suspended in 50 mL of methanol. The suspension was exposed to ultrasonic waves for 5 minutes and stirred using a magnetic stirrer for 30 minutes, and then methanol was added thereto until the amount of the resultant solution was 100.0 mL. The obtained solution was left to sit for 10 minutes. Methanol was added to 5.0 mL of the left to sit solution until the total amount of the solution was 50.0 mL. The resultant solution was filtered through a 0.45 μm micropore membrane. At this point, 5 mL of the initially filtered solution was left unused. The content analysis was performed under chromatography conditions as shown in Table 2.

The results are shown in Table 4.

TABLE 4

| | Initia- | | | | |
|---|---|---|---|---|---|
| | tion | 1 week | 2 weeks | 3 weeks | 4 weeks |
| Plavix | 100 | 96.83 | 89.59 | 78.42 | 81.72 |
| Comparative Example 1 | 100 | 93.20 | 90.03 | 86.22 | 85.32 |
| Example 1 | 100 | 94.98 | 93.76 | 93.05 | 90.52 |
| Comparative Example 2 | 100 | 94.80 | 89.20 | 87.13 | 84.41 |
| Comparative Example 3 | 100 | 98.83 | 87.69 | 85.72 | 61.17 |
| Example 2 | 100 | 97.17 | 93.56 | 88.36 | 87.89 |
| Comparative Example 4 | 100 | 96.81 | 92.29 | 83.12 | 75.82 |

According to the results shown in Table 4, the tablets comprising pregelatinized starch prepared according to Examples 1 and 2 have a higher stability than Plavix and the tablets prepared according to Comparative Examples 1 through 4.

1. A pharmaceutical composition comprising clopidogrel bisulfate and pregelatinized starch.

2. The pharmaceutical composition of claim 1, wherein the amount of clopidogrel bisulfate is in the range of 30-40 parts by weight and the amount of the pregelatinized starch is in the range of 10-70 parts by weight.

3. The pharmaceutical composition of claim 1, wherein the clopidogrel bisulfate is Crystalline Form I or Crystalline Form II.

4. The pharmaceutical composition of claim 1, wherein the pregelatinized starch is selected from the group consisting of pregelatinized corn starch, pregelatinized potato starch, pregelatinized wheat starch, and a mixture thereof.

5. A tablet formed of the pharmaceutical composition of claim 1.

6. A tablet formed of the pharmaceutical composition of claim 2.

7. A tablet formed of the pharmaceutical composition of claim 3.

8. A tablet formed of the pharmaceutical composition of claim 4.
